(19) United States
(12) Patent Application Publication
(10) Pub. No.: US 2008/0214996 A1
(43) Pub. Date: Sep. 4, 2008

(54) NEEDLELESS INJECTORS

(76) Inventors: STEVEN D. KIMMELL, Granada Hills, CA (US); Scott J. Gerondale, Mission Viejo, CA (US)

Correspondence Address: Allergan, Inc. (T2-7H), 2525 Dupont Drive, Irvine, CA, 92612, US

(21) Appl. No.: 12/107,486

(22) Filed: Apr. 22, 2008

Related U.S. Application Data
(62) Division of application No. 10/557,105, filed on Nov. 14, 2005.
(60) Provisional application No. 60/480,320, filed on Jun. 20, 2003.
(60) Provisional application No. 60/480,784, filed on Jun. 23, 2003.
(60) Provisional application No. 60/491,159, filed on Jul. 29, 2003.
(60) Provisional application No. 60/497,992, filed on Aug. 26, 2003.
(60) Provisional application No. 60/494,463, filed on Aug. 11, 2003.
(60) Provisional application No. 60/497,780, filed on Aug. 26, 2003.
(60) Provisional application No. 60/480,665, filed on Jun. 23, 2003.
(60) Provisional application No. 60/484,069, filed on Jul. 1, 2003.

(51) Int. Cl. A61M 5/30

(52) U.S. Cl. 604/68

(57) ABSTRACT

A needleless injector generally includes a barrel having a proximal and a distal end and a bore therethrough along with an injection head disposed at the barrel distal end in fluid communication with the bore; an energy source is disposed at the barrel proximal end and a bolt may be disposed in said bore for reciprocating movement therein, from a first position to a second position for drawing medicament into the bore, the bolt being fitted in the bore in order to force medicament through the injection head upon movement of the bolt from the second to the first position. A mechanism is provided for activating the energy source in order to operate the bolt.

### CROSS REFERENCE TO RELATED APPLICATIONS

The present application is a division of U.S. Ser. No. 10/557,105 which is a national stage application under 35 U.S.C. § 371 of PCT application PCT/US2004/019491, filed on Jun. 17, 2004, which claims the benefit of Provisional Application Nos. 60/480,320, filed on Jun. 20, 2003; 60/480,784, filed Jun. 23, 2003; 60/491,159, filed Jul. 29, 2003; 60/497,992, filed Aug. 26, 2003; 60/494,463, filed Aug. 11, 2003; 60/497,780, filed Aug. 26, 2003; 60/480,665, filed Jun. 23, 2003; and 60/484,069; filed Jul. 2, 2003.

### FIELD OF THE INVENTION

The present invention is generally directed to medicament injectors and is more particularly directed to needleless injectors.

A great number of injectors have been developed for transdermally providing medicament doses. All of these devices require sufficient energy to propel a medicament through the dermal surface of a patient. Such energy may be provided through the use of mechanical leverage, springs, compressed gas, or electrical solenoids.

Injectors may be developed for single use or have a rechargeable medicament and energy storage system. All of these systems require sufficient force on a piston or the like to eject all of the medicament at a pressure sufficient for dermal penetration.

The present invention is directed to needleless injectors for transdermal delivery of a medicament using a piston/or bolt through an injection head by an energy source.

### SUMMARY OF THE INVENTION

A needleless injector in accordance with the present invention generally includes a housing, casing, or barrel along with an injection head and a piston, mold, or cylinder for forcing a medicament through the injection head upon the release of actuating energy.

### BRIEF DESCRIPTION OF THE DRAWINGS

The present invention may be more clearly understood with reference to the appended drawings of which:

FIG. 1 is a perspective view of a first embodiment of the present invention generally showing a housing, an injection head, and a vial along with a trigger;

FIG. 2 is a side view and partial cross section of the mechanism disposed within the housing, as shown in FIG. 1, which generally includes, an injection head, a vial, a bolt, a cocking stud, a valve tube, a mainspring, a hammer, a sear, a valve seat, a trigger, a cup seal, a rear spring, and a gas cartridge;

FIG. 3 is a cross sectional view of the injector shown in FIG. 2 with the bolt shown in a cocked position ready for injection of medicament through an injection head;

FIG. 4 is a view similar to that shown in FIG. 3 with the bolt shown in an unlocked position after dispensing of the medicament through the injection head;

FIG. 5 is a front perspective view of a second embodiment in accordance with the present invention which includes a revolver needleless injector generally showing a frame, barrel, vial, a cylinder for supporting a plurality of cartridges, and a trigger mechanism;

FIG. 6 is a rear perspective view of the injector shown in FIG. 5;

FIG. 7 is a cross sectional view of the injector shown in FIG. 5 showing a port near the barrel distal end, a bolt disposed within the barrel bore along with a hammer, and a spring mechanism, a bolt being shown in a second position before firing of a cartridge and ejection of medicament through the injection head;

FIG. 8 is a cross sectional view similar to that shown in FIG. 7 illustrating depression of the trigger and movement of the gas cartridge aligned with the hammer and bore driving the bolt forward, as will be hereinafter described in greater detail;

FIG. 9 is a view taken along the line 9-9 of FIG. 7;

FIG. 10 is a perspective view of a third embodiment in accordance with the present invention generally showing a tubular casing having a bottom half shell and a top half shell;

FIG. 11 is a perspective view similar to that shown in FIG. 10 showing the top half shell in an open position during cocking of a spring shown disposed within the tubular casing;

FIG. 12 is a cross sectional view of the needleless spring injector as shown in FIGS. 10 and 11 showing a spring in a cocked position, a plunger ratchet, a push rod, syringe, and trigger;

FIG. 13 is a cross sectional view similar to that shown in FIG. 12 illustrating movement of the ratchet one step causing forward motion of the plunger by the spring and injection of medicament through the injection head;

FIGS. 14-18 illustrate the cocking mechanism of the third embodiment, which is effected through hinged opening of the top half shell of the injector;

FIG. 19 is a perspective view of a fourth embodiment of a needleless push release injector in accordance with the present embodiment showing a housing and cocking ring;

FIG. 20 is a perspective view of the embodiment shown in FIG. 19 illustrating internal components, including a vial, check valve, piston, spring, along with the cocking ring and injection head;

FIG. 21 is a cross sectional view of the injector shown in FIGS. 19-20, more particularly illustrating components of the present invention in which the spring is held in a compressed state by a sear prior to release for injection of medicament through an injection head into the patient's skin, not shown in FIG. 21;

FIG. 22 is a cross sectional view similar to that shown in FIG. 21 illustrating release of the piston upon pressure being applied to the injection head by the skin of a user;

FIG. 23 is a perspective view of the injection head, cylinder, piston, and cocking ring;

FIG. 24 is a perspective view of a fifth embodiment of the present invention which includes a push-pull injector generally showing a housing, a vial for containment of a medicament, an injector head, a cocking grip, and trigger;

FIG. 25 is a cross sectional view of the injector shown in FIG. 24 more specifically showing a chamber disposed within the housing, a piston slidably disposed within the housing, and a piston head fitted within the chamber, and having a stem with a spring disposed there around along with a sear for holding a piston in a second position with the spring compressed;

FIG. 26 is a cross sectional view similar to FIG. 25 showing release of the piston with the spring forcing the piston head through the chamber in order to force a medicament through the injection head;

FIG. 27 is a cross sectional view similar to FIGS. 25 and 26 showing cocking of the spring and withdrawal of the medicament from the vial into the chamber for subsequent injection;

FIG. 28 is a perspective view of a sixth embodiment in accordance with the present invention which includes a twist-set injector generally showing a housing, an injection head, a rotatable grip, and a trigger;

FIG. 29 is a plan view of the injector shown in FIG. 28 partially broken away to illustrate a vial for containment of a medicament, a piston, a one-way valve, a mainspring, and a check valve;

FIG. 30 is a cross sectional view of the injector shown in FIGS. 28 and 29 more specifically showing a chamber disposed within the housing, a piston slidably disposed within the housing with a piston head fitted within the chamber and having a stem with a spring disposed there around along with a sear for holding the piston in a second position with a spring compressed;

FIG. 31 is a cross sectional view similar to FIG. 30 showing release of the piston with the spring forcing the piston head through the chamber in order to force medicament through the injection head;

FIG. 32 is a cross sectional view similar to FIGS. 30 and 31 showing cocking of the spring and withdrawal of medicament from the vial into the chamber for subsequent injection by rotation of the grip;

FIG. 33 is a perspective view of a seventh embodiment of the present invention which shows an inline needleless injector and generally showing a casing, an injection head disposed at a casing distal end, and a gas line;

FIG. 34 is a view of a sub-embodiment in accordance with the present invention and partial cross section generally showing a hollow casing having a chamber disposed therein along with a piston, gas manifold, drug manifold, vial, duckbill valve, and cocking stud;

FIG. 35 is a cross sectional view of a second sub-embodiment of the present invention generally showing a chamber piston bias by a spring; and

FIG. 36 is a cross sectional view corresponding to the embodiment shown in FIG. 35 with the application of gas pressure for driving a piston forward for forcing medicament through the injection head;

FIG. 37 is a perspective view of an eighth embodiment of a needleless solenoid driven injector in accordance with the present invention generally showing a housing, an injection head, and an attached power cord;

FIG. 38 is a cross sectional view of the injector shown in FIG. 37 illustrating a cylinder, a piston disposed therein along with a solenoid driven plunger operated by a trigger, check valves for controlling flow of medicament through a manifold, and dip tub for withdrawal of medicament from a vial disposed within the housing; and

FIGS. 39 and 40 are cross sectional views illustrating operation of the needleless solenoid driven injector shown in FIGS. 37, 38 illustrating operation of the needleless solenoid driven injector in accordance with the present invention.

### DETAILED DESCRIPTION

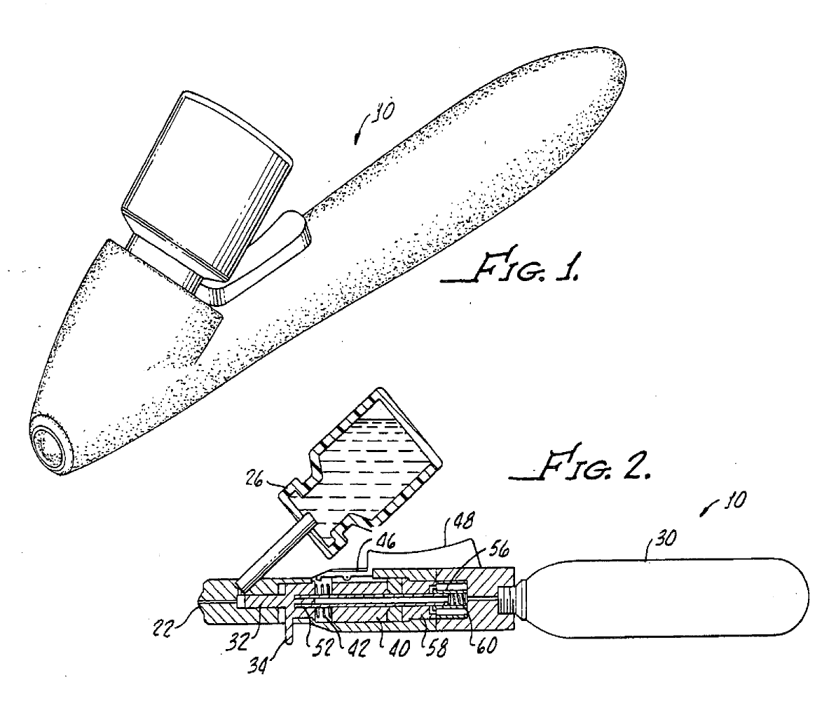

With reference to FIGS. 1-4, there is shown a first embodiment of a needle injector 10 in accordance with the present invention which generally includes a barrel 12 having a proximal end 14 and a distal end 16 with a bore 20 therethrough.

An injection head 22 is disposed at the barrel distal end 16, which is in fluid communication with the bore 20 for the injection of a medicament therethrough.

A vial 26 is provided for containing a medicament, such as BOTOX® (botulinum toxin), with the vial being in fluid communication with the bore 20 near the barrel distal end 16. Preferably, the vial 26 is removably attached to the barrel 12.

A gas cartridge 30 is disposed at the barrel proximal end 14 and a bolt 32 is provided and disposed in the bore 20 for reciprocal movement therein from a first position closing the port 28 to a second position opening the port for release of medicament into the bore 20.

The bolt 32 is sized and fitted to provide sufficient seal between the bolt 32 and the bore 20 in order to draw medicament from the vial into the bore 20 upon movement of the bolt 32 from the first position to the second position and to force medicament through the injection head 22 upon movement of the bolt 32 from the second position to the first position, as will be hereinafter described in greater detail.

A cocking stud 34 is provided for enabling manual movement of the bolt 32 from the first and second position. In this cocking movement, medicament is drawn into the bore 20 between the bolt 32 and the injection head 22 through the port 28.

A hammer 40 is provided and disposed for reciprocal movement within the bore 20 between the barrel and the proximal and distal end 12, 14. A mainspring 42 is disposed between the bolt 32 and the hammer 40 for forcing the hammer forwardly toward the barrel distal end 16.

A sear 46 is provided for releasably holding the bolt 32 in the second position with the mainspring 42 compressed between the bolt 32 and the hammer 40 and a trigger 48 is disposed in an operative relationship with the sear 46 for releasing the spring 42 in order to drive the hammer 40 toward the barrel distal end 16.

A valve tube 52 is provided and disposed within the bore 20 with the distal thereof attached to the bolt 32 and a proximal end attached to the gas cartridge 30. The valve tube 52 extends through the hammer 40 and mainspring 42.

A cup seal 56 includes a valve seat 58 for controlling gas flow from the cartridge 30 into the valve tube 52.

In that regard, a rear spring 60 is provided for releasably sealing the cup seal 56 against the valve seat 58. The cup seal 56 is open momentarily by impact of the hammer 40 after release by the sear 46 in order to allow gas to flow through the valve tube 52 to force the bolt 32 to the first position which ejects medicament through the injection head 22. The rear spring 60 thereafter closes the cup seal 56 against the valve seat 58 after the momentary opening.

In operation, when the bolt 32 is not cocked, a forward portion blocks entry of the medicament into the bore 20 extending through the barrel 12. In order to cock the injector 10, the cocking stud 34 is moved rearwardly, pushing against the mainspring 42 in order that the bolt 32 butts up against the hammer 40. This motion opens the port 28 between the vial 26 and the bore 20 and draws medicament into the bore 20 between the injection head 22 and the bolt 32. The sear 46 catches the bolt 32 as the bolt 32 is pushed against the hammer 40 and this binds the bolt 32 and the hammer 40 together so they move as one unit.

For injecting a medicament, the trigger 48 is pushed in order to release the sear 46 which releases the hammer 40 from the bolt 32 and the mainspring 42 propels the hammer 40 rearwardly in order to push momentarily onto the valve tube 52 which propels the valve backward with sufficient force to overcome the rear spring 60. At that point, gas pressure is released from the gas cartridge 30 through the tube 52, which causes the bolt 32 to rapidly move and eject medicament disposed between the front of the bolt 32 and the injection head 22 through the injection head 22.

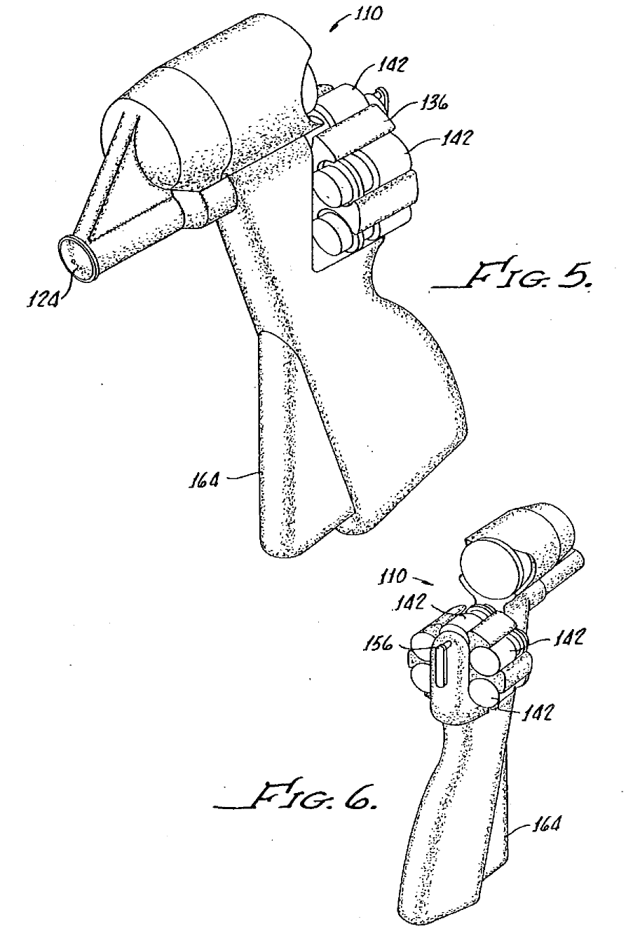

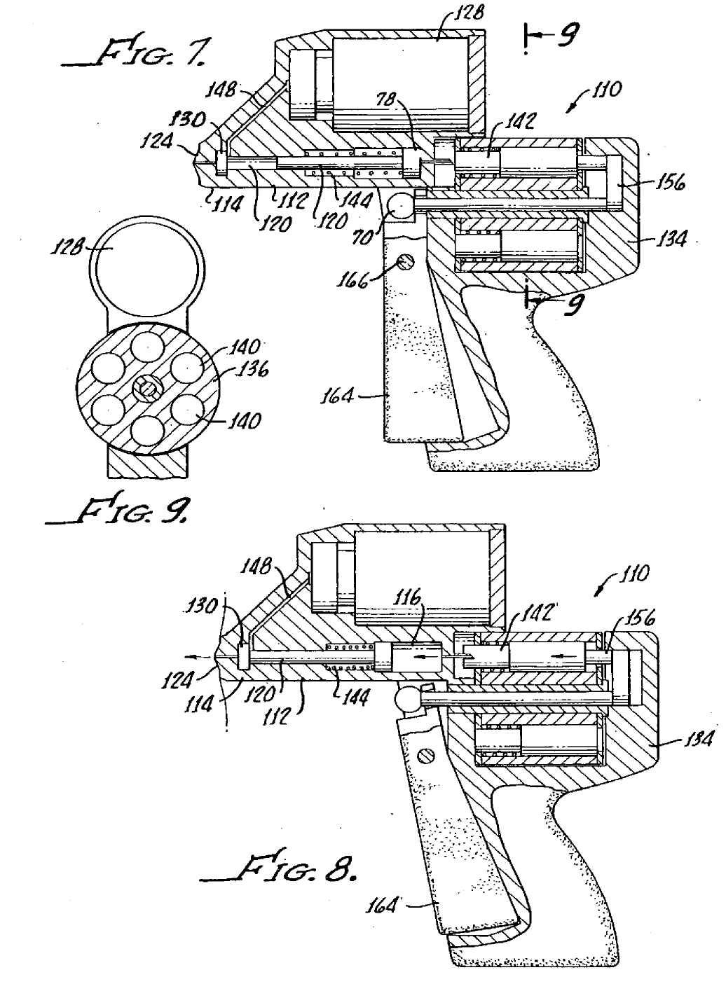

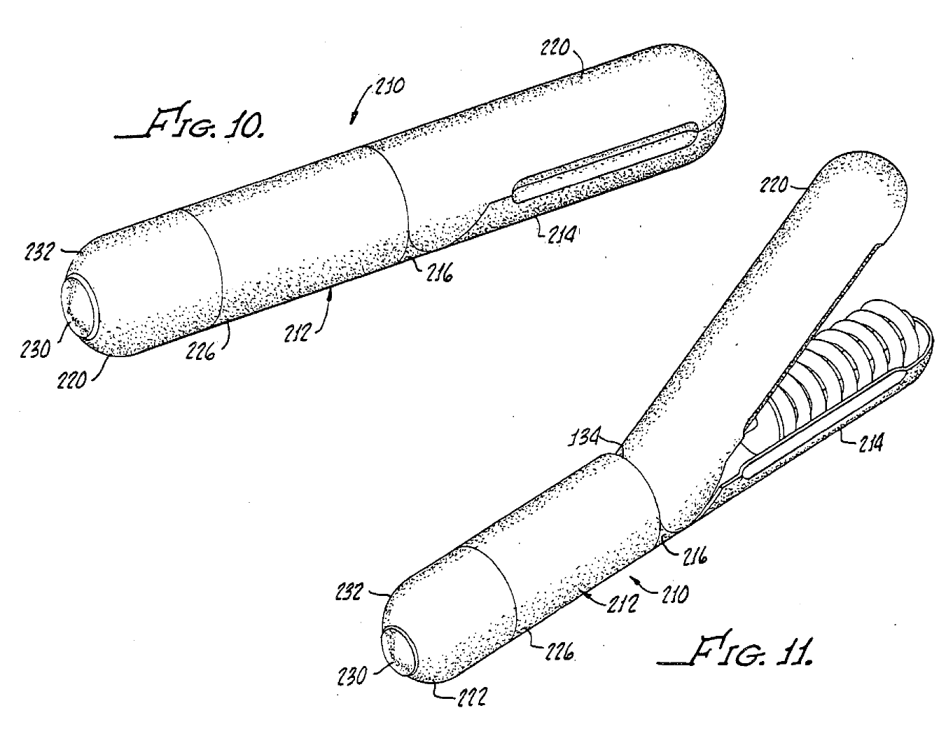

With reference to FIGS. 5-9 a second embodiment of a needleless injector 110 in accordance with the present invention generally includes a barrel 112 having a proximal end 114 and a distal end 116 with a bore 120 therethrough. An ejection head 124 is disposed at the barrel distal end 114 which is in fluid communication with the bore 120 for the ejection of a medicament therethrough.

A vial 128 is provided for containing a medicament, such as BOTOX® (botulinum toxin), with the vial 128 being in fluid communication with the bore 120 through a port 130 near the barrel distal end 114. Preferably the vial 128 is removably attached to the barrel 112 which is in turn supported by a frame 134.

A cylinder 136 is rotatably supported by the frame 134 at the barrel proximal end 114 and includes a plurality of chambers 140 therein for receiving gas cartridges 142.

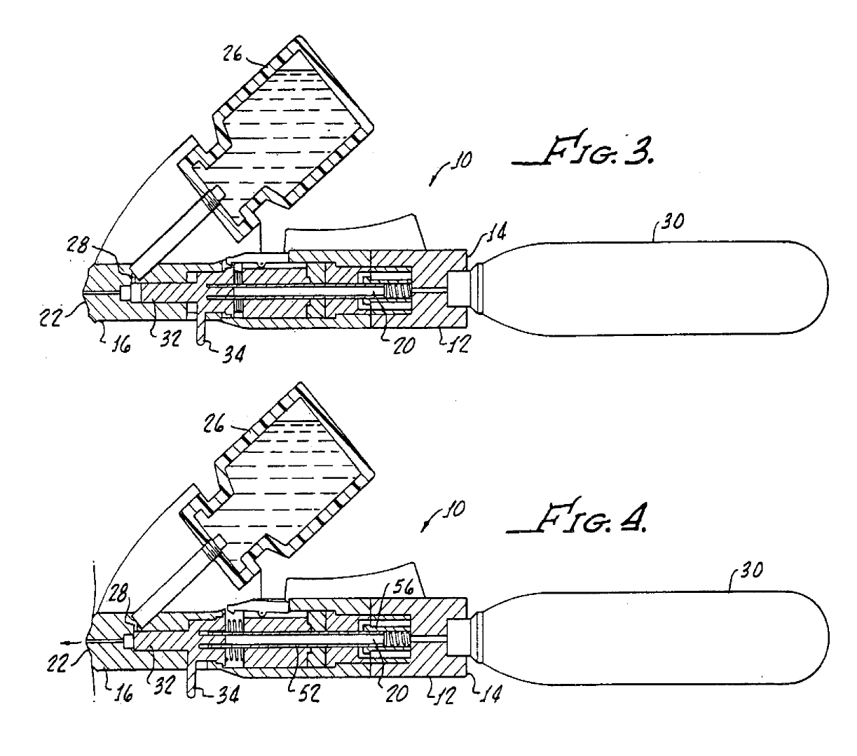

A bolt 144 is disposed in the bore 120 for reciprocating movement from a first position closing a port 130 as shown in FIG. 4 to a second position as shown in FIG. 3 opening the port 130 for release of medicament into the bore.

The bolt 144 is fitted within the bore 120 in order to draw medicament from the vial 128 through the port 130 via a line 148 upon movement of the bolt 144 from the first position to the second position. Movement of the bolt 144 from the second to the first position as hereinafter described forces a medicament through the injection head 124. The injection head may be of any conventional design.

A spring 152 is disposed adjacent to the bolt 144 for driving the bolt 144 from the first to the second position, as hereinafter described.

A firing pin 160 may be disposed between the bolt 144 and the cylinder 136 for puncturing a gas cartridge 142 aligned with a hammer 156 and bore 120 by rotation of the cylinder 136. A trigger 164 pivotally 166 mounted to the frame 134 utilizes a linkage 170 for forcing the aligned cartridge 142 with the hammer 156 for puncture of the cartridge 142 in order to pneumatically drive the bolt 144 from the second position to the first position and force medicament in the bore through the ejection head 124, as shown in FIG. 4. After release of pressure, the spring 112 forces the bolt 144 from the first position to the second position, thus drawing a medicament into the bore 124 for repeated injection.

It should be appreciated that the cylinder 136 may be manually rotated to align bore 120 and hammer 156 or a pawl and ratchet arrangement may be provided in order to rotate the cylinder 136 prior to a movement of a gas cartridge 142 into engagement of the hammer for release of pressure therein.

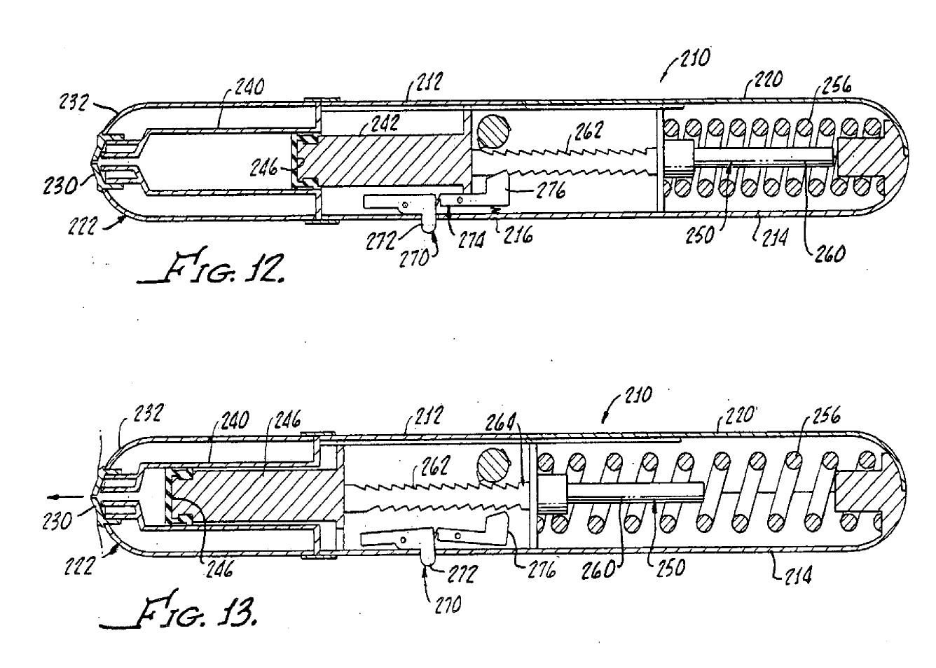

With reference to FIGS. 10 and 11, a third embodiment of a needleless spring injector 210 in accordance with the present invention generally includes a tubular casing 212 having a bottom half shell 214 extending from a rear end 216 of the tubular casing 212.

A top half shell 220 is hingeably attached to the casing rear end 214 for covering the bottom half shell 214.

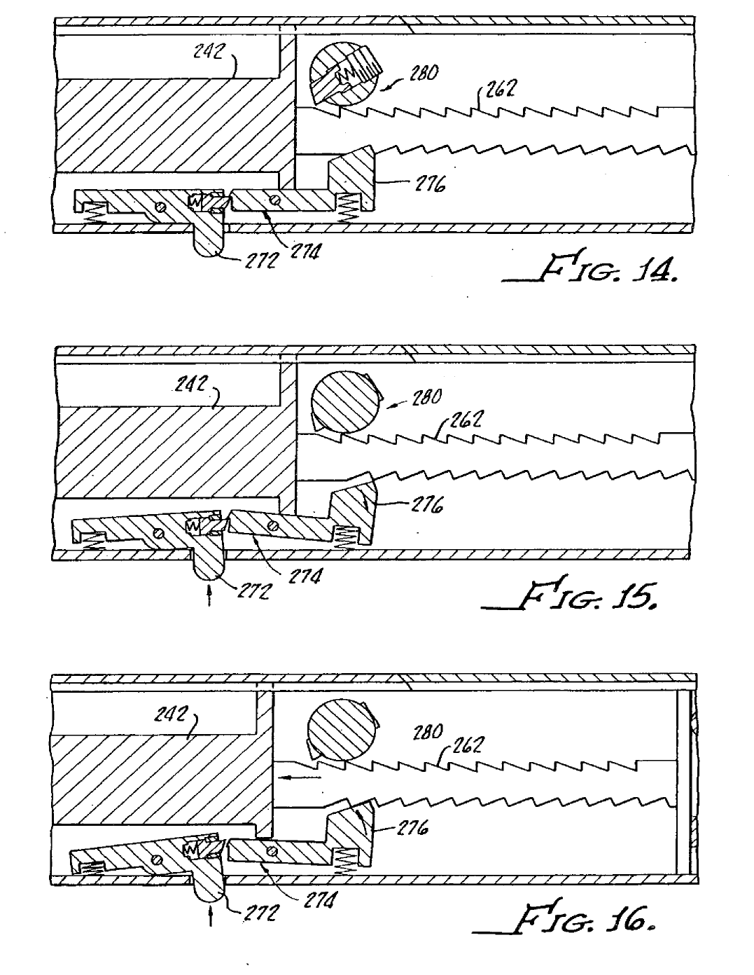

A nose cap 222 is removably attached to a front end 226 of the tubular casing 212 and an injection head 230 is disposed in an end 232 of the nose cap 222. As more clearly shown in FIGS. 12 and 13, a syringe 240 may be replaceably disposed within the tubular casing 212 which is in fluid communication with the injection head 230. A plunger 242 is slidably disposed within the tubular casing 212 and engages one end 246 of the syringe 240 for ejecting medicament therefrom through the injection head 230.

A push rod is housed between the top half shell 220 and bottom half shell 214 is provided for slidably moving the plunger 242 and a spring 256 is disposed about a rear portion 260 of a push rod 250 for driving the push rod.

A ratchet 262 is disposed on a forward portion 264 of the push rod and manually operated trigger 270 which is pivotably mounted within the tubular casing 212 includes a button extending outwardly from the tubular casing 212 for manual depression of a link mechanism 274 and a sear 276 for releasably engaging a ratchet 262.

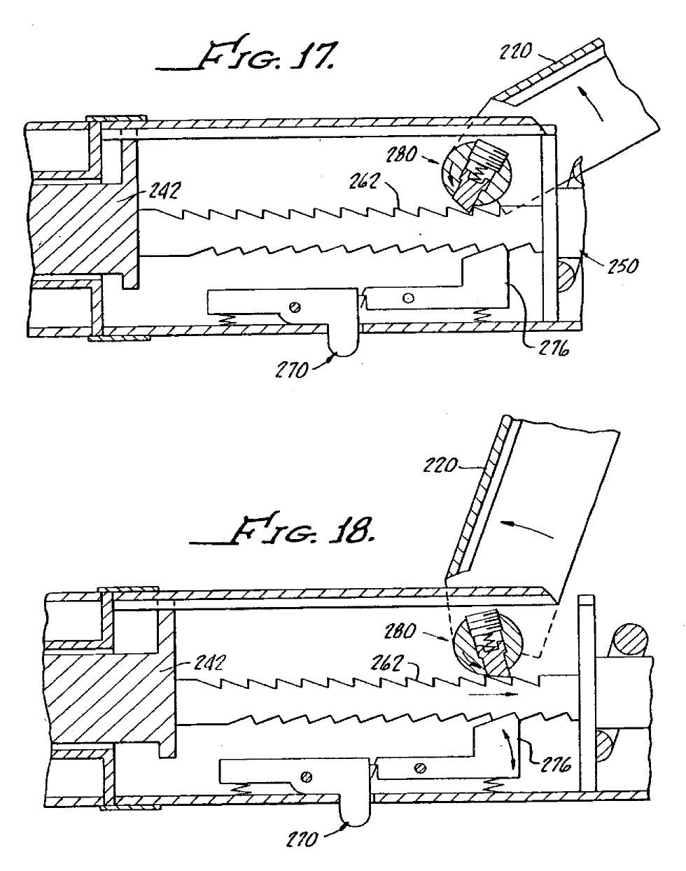

FIGS. 14-16 illustrate the depression of the button 272 initiating a single step forward movement of the ratchet 220 and push rod 250 for single dose ejection of a medicament, such as, BOTOX® (botulinum toxin).

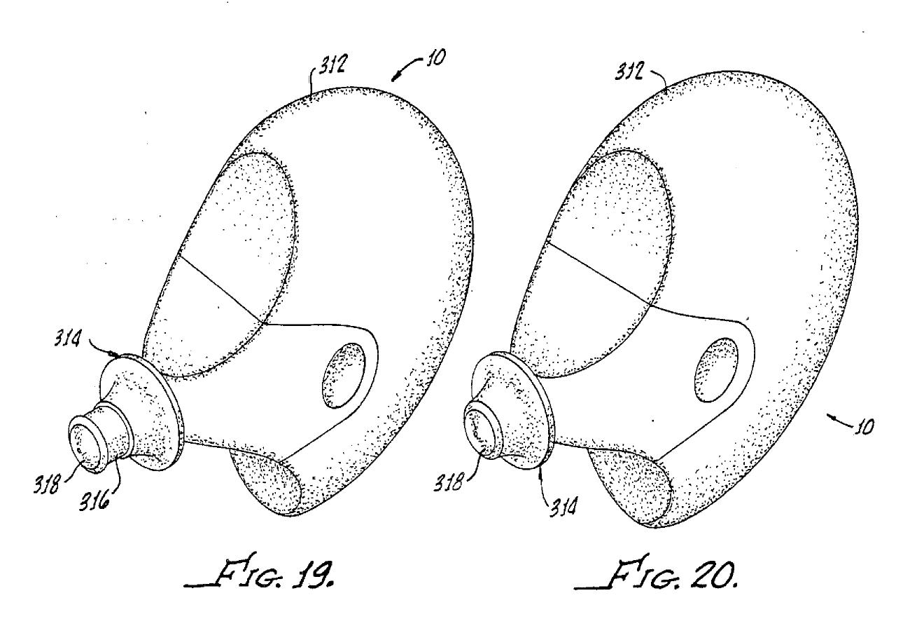

FIGS. 17 and 18 show a cocking mechanism 280 for engaging the push rod ratchet 262 and compressing the spring 256 on hinged opening of the top half shell 220 from the bottom half shell 214. The spring 256 to retain in a compressed state by engagement of the sear 276 with the ratchet 262.

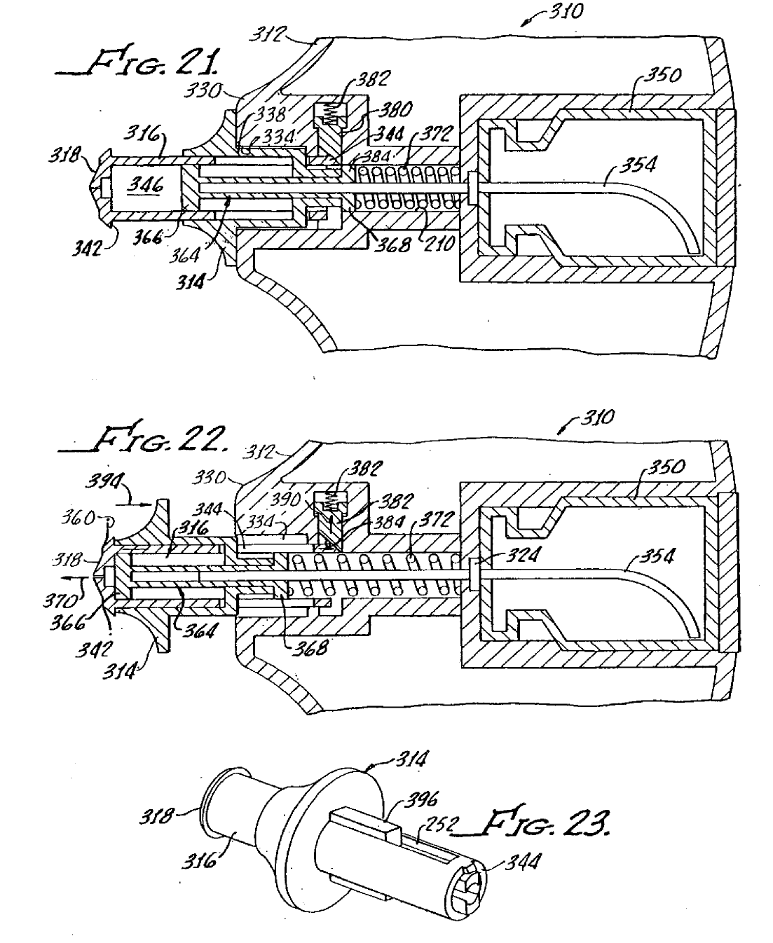

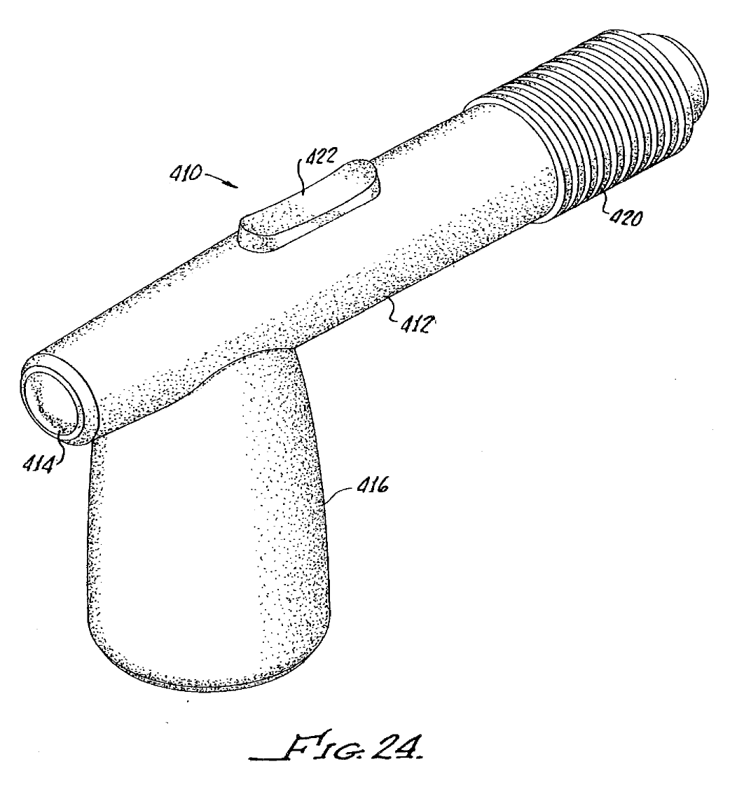

With reference to FIGS. 19 and 20 there is shown a fourth embodiment of a needleless push-release injector 310 in accordance with the present invention generally showing a housing 312 along with a cocking ring 314, a cylinder 316, injection head 318, a vial 320, piston 322, valve 324, and a spring 326. With reference to FIGS. 21 and 22, a housing 312 includes a distal end 330 and a proximal end 332 with a bore 334 therein. The cocking ring 314 extends from the housing distal end 330 and includes a rear portion 338 disposed within the housing bore 334.

The cylinder 316 is slidably disposed within the cocking ring 314 and includes a front end 342 and a rear end 344 with a chamber 346 disposed proximate the front end 342. A vial 350 for containing a medicament is replaceably disposed proximal end 332 and is in fluid communication with the chamber 346 through a dip tube 324 and check valve 324.

The injection head 318 is disposed at the cylinder front end 342 and in fluid communication with the chamber 346. The injection head may be of any suitable design.

A piston 364 is slidably disposed within the cocking ring 314 and includes a front head 366 slidably disposed within the cylinder 316 and a rear head 368 slidably disposed within the housing bore 334.

The front head 366 is fitted to the cylinder 316 in order to draw medicament from the vial 350 into the chamber through the one-way valve 324 upon movement of the piston 364 from a first position shown in FIG. 22 to a second position shown in FIG. 21. Movement of the piston from the first position to the second position forces medicament from the chamber 346 through the injection head 318.

A main spring 372 is disposed between the housing proximal end 332 and the piston rear head 368 and a sear 380 is provided for releasably holding of the piston 364 in the second position with the main spring 372 compressed. The sear 380 includes a sear spring 382 and a tapered end 384 which provides releasable engagement with the piston rear head 368.

In use, the injector head 318 is pressed against the skin 360 which causes rearward motion of the cylinder 316 and the cylinder rear end 344 is urged against the tapered end 384 causing movement of the sear in the direction of the arrow 390 and compression of the sear spring 382 which releases the piston rear head 368 and piston 364, thus causing the spring 372 to force the piston front head 366 into the chamber 346 forcing medicament through the injection head 318, as indicated by the arrow 370.

To re-cock the device the cocking ring 314 is moved in the direction of the arrow 394, thus moving the piston end 384 and re-engagement of the sear 380 with the piston rear head 368.

As illustrated in FIG. 23, the cocking ring may include guides 396 for preventing rotation thereof.

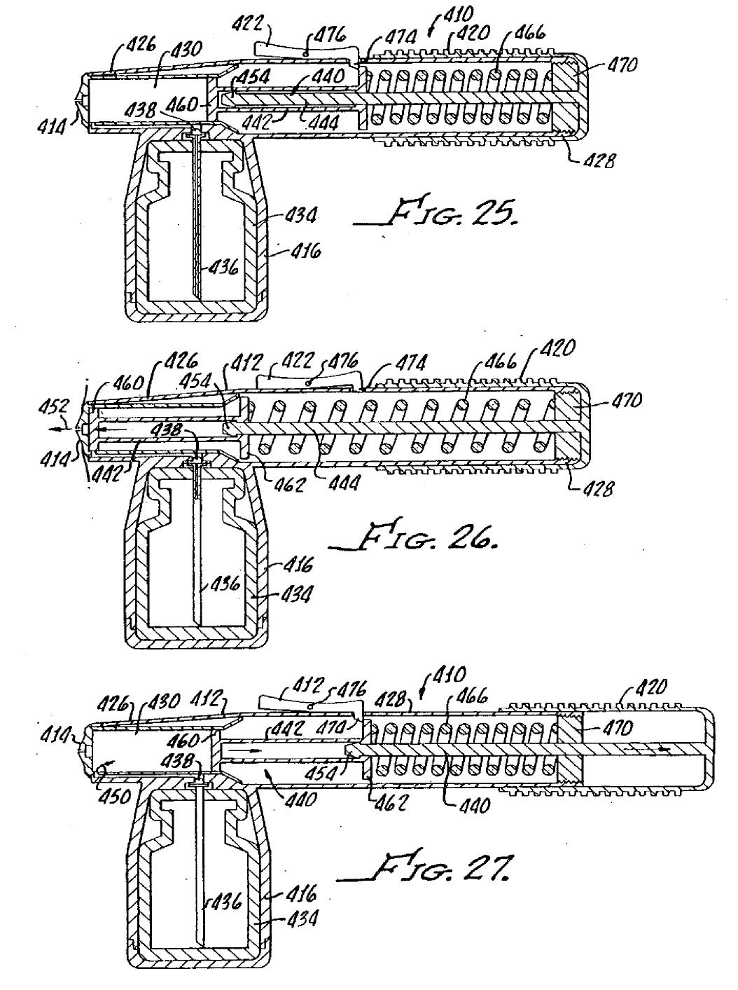

With reference to FIG. 24, there is shown a fifth embodiment of a push-pull needleless injector 410 generally including a housing 412, an injection head 414, a vial 416 for containing a medicament, such as, for example, BOTOX® (botulinum toxin), a cocking grip 420 and a trigger 422.

As shown in FIGS. 25-27, the housing 412 includes a distal end 426 and a proximal end 428 with a chamber 430 disposed within the housing 412 at the distal end 426.

The injection head, which may be of conventional design, is disposed at the housing distal end 426 and is in fluid communication with the chamber 430.

The vial 416 may support a replaceable container 434 containing a medicament, not shown, as hereinabove referenced and is in fluid communication with the chamber through a dip tube 436 and a one-way valve 438.

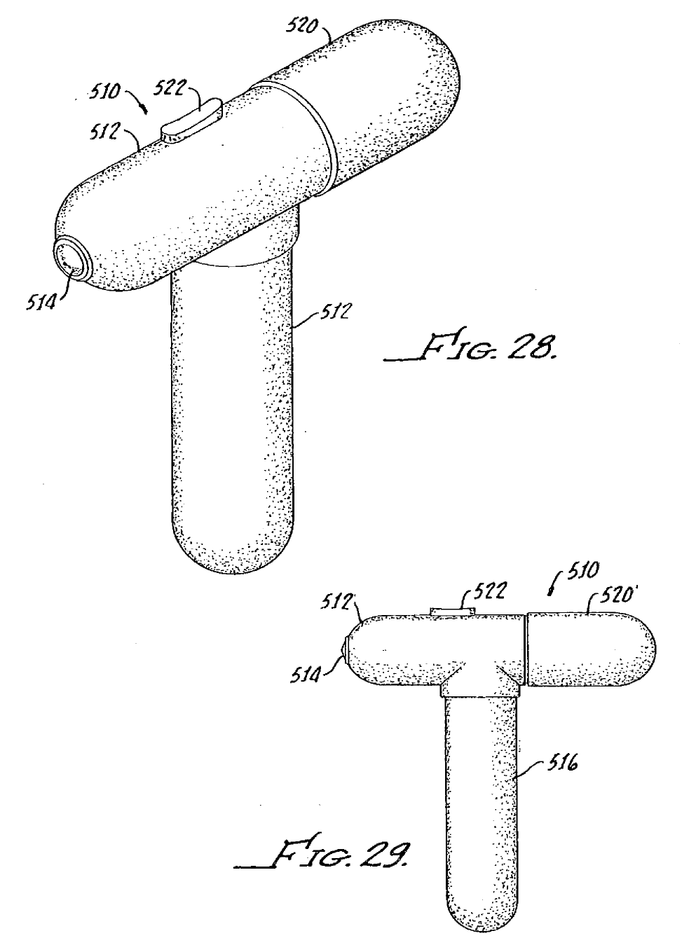

A piston 440 is slidably disposed within the housing 412 and includes a piston head slidably disposed within the chamber 430 and a separately moveable stem 444 disposed in the housing 428. The piston head 442 is fitted within the chamber 430 in order to draw medicament from the vial 416 into the chamber 430 through the one-way valve 438 and dip tube 436 upon movement of the piston head from a first position, as shown in FIG. 26, to a second position shown in FIG. 27 the entry of fluid into the chamber being indicated by the arrow 450 in FIG. 27.

Medicament is forced through the injection head 414 by movement of the piston head 442 from the second position, as shown in FIG. 25, to the first position as shown in FIG. 26 with the injected medicament being indicated by the arrow 452. A head 454 on the piston stem 444 couples the stem 444 in a slidable manner with the piston head 442 between a fore plate 460 an aft plate 462 of the piston head 442.

As shown in FIGS. 25-27, the spring 466 disposed around the piston stem 444 between the aft plate 462 and an end plate 470 which is compressed by the cocking grip 420 which is slidably disposed over the housing proximal end 428. Reciprocal movement of the cocking grip causes compression of the spring, as illustrated in the figures. A sear is provided for releasably holding the piston 444 in the second position with the spring 466 compressed by engagement with the aft plate 462, as shown in FIGS. 26 and 27.

The trigger 422 is pivotally 476 disposed on a housing 412 and in an operational relationship with the sear 474 for releasing the spring 466 in order to drive the piston 442 along with fore plate 460 to the first position, thus ejecting a metered dose of medicament determined by the chamber 430 volume.

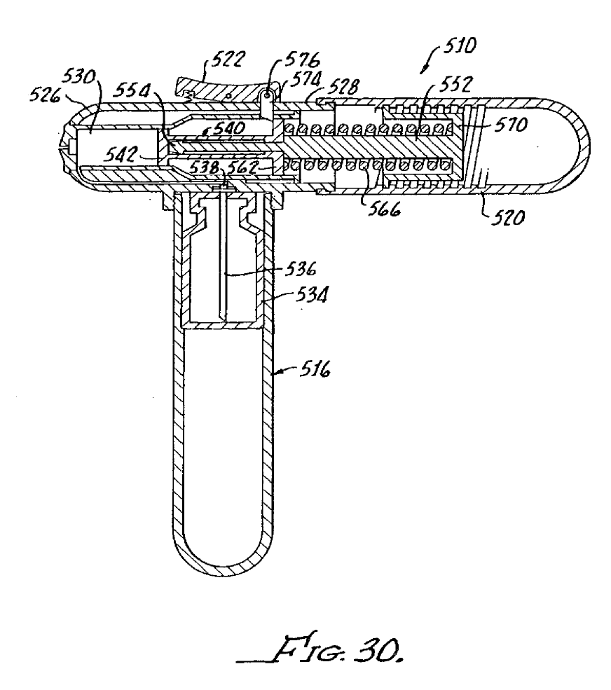

With reference to FIGS. 28 and 29, there is shown a sixth embodiment or twist-set needleless injector 510 generally including a housing 512, an injection head 514, a vial 516 for containing a medicament, such as, for example, BOTOX® (botulinum toxin), a rotatable grip 520 and a trigger 522.

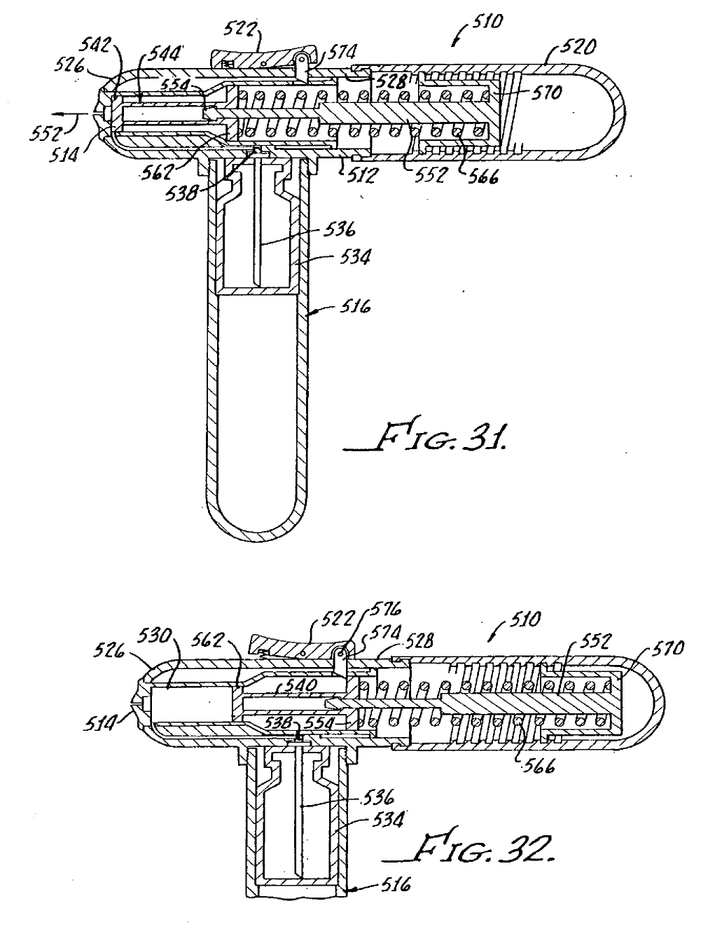

As shown in FIGS. 30-32, the housing 512 includes a distal end 526 and a proximal end 528 with a chamber 530 disposed within the housing 512 at the distal end 526.

The injection head 514, which may be of conventional design, is disposed at the housing distal end 526 and is in fluid communication with the chamber 530.

The vial 516 may support a replaceable container 534 containing a medicament, not shown, as hereinabove referenced and is in fluid communication with the chamber through a dip tube 536 and a one-way valve 538, for example a duckbill valve.

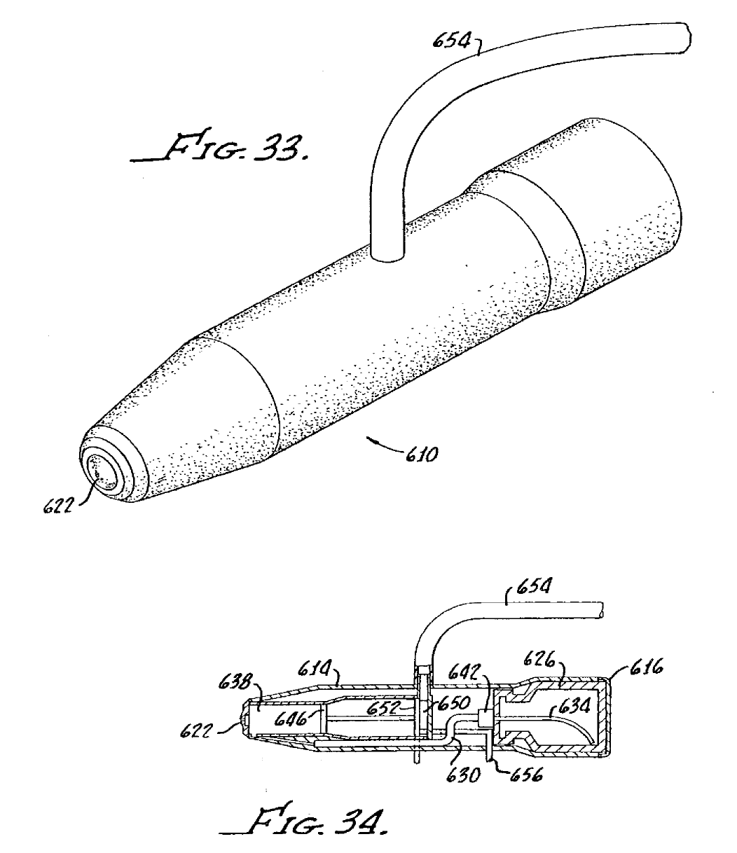

A piston 540 is slidably disposed within the housing 512 and includes a piston head 542 slidably disposed within the chamber 530 and a moveable stem 544 disposed in the housing 512. The piston head 542 is fitted within the chamber 530 in order to draw medicament from the vial 516 into the chamber 530 through the one-way valve 538 and dip tube 536 upon movement of the piston head from a first position, as shown in FIG. 31, to a second position shown in FIG. 32 the entry of fluid into the chamber being indicated by the arrow 550 in FIG. 32.

Medicament is forced through the injection head 514 by movement of the piston head 542 from the second position, as shown in FIG. 31, to the first position as shown in FIGS. 30 and 32 with the injected medicament being indicated by the arrow 552. A head 554 on the piston stem 544 couples the stem 544 in a slidable manner with the piston head 542 between a fore plate 560 an aft plate 562.

As shown in FIGS. 30-32, a spring 566 disposed around the piston stem 544 between the aft plate 562 and an end plate 570 which is compressed by the rotating grip 520 which is threadably disposed at the housing proximal end 528. Rotational movement of the cocking grip causes compression of the spring 566, as illustrated in the figures. A sear 574 is provided for releasably holding the piston 544 in the second position with the spring 566 compressed by engagement with the aft plate 562, as shown in FIG. 30.

The trigger 522 is pivotally 576 disposed on the housing 512 and in an operational relationship with the sear 574 for releasing the spring 566 in order to drive the piston 542 along with fore plate 560 to the first position, thus ejecting a metered dose of medicament determined by the chamber 530 volume.

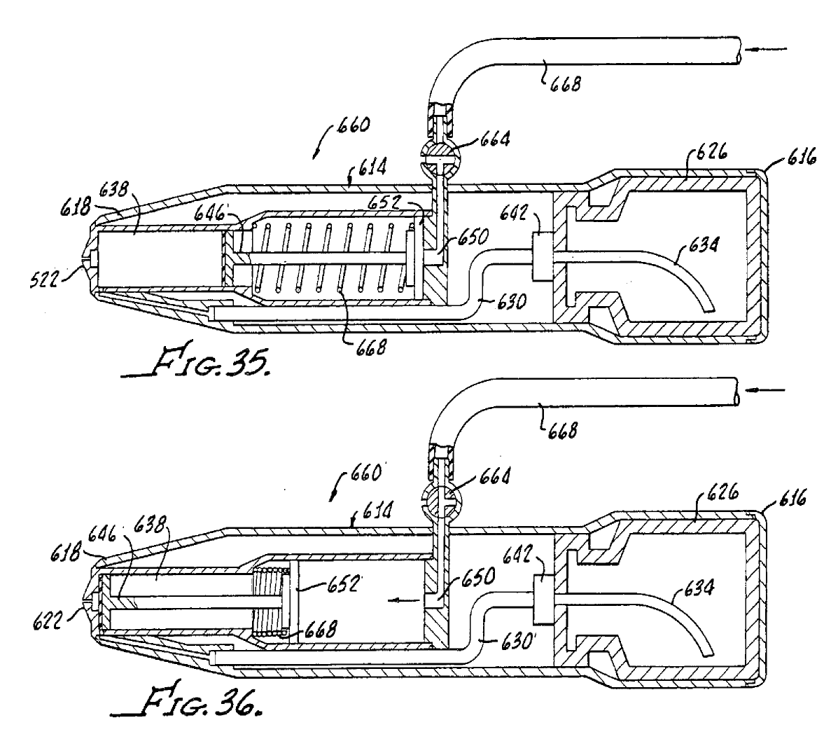

With reference to FIGS. 33 and 34, there is shown a seventh embodiment or inline needleless injector 610 having a hollow casing 614 with a proximal end 616 and a distal end 618, an injection head 622 is disposed at the casing distal end 618 and a vial 626 for containing a medicament, such as, for example, BOTOX® (botulinum toxin), is disposed within the casing 614 at the proximal end 616 thereof.

A drug manifold or line 630 communicates with the vial 626 through a dip tube 634 and a chamber 638 for transport of medicament from the vial 626 into the chamber 638. A one-way valve 642, such as, for example, a duckbill valve, disposed in the drug manifold 630 prevents transport of medicament into the vial from the chamber.

A piston 646 is provided and slidably disposed within the chamber for forcing medicament through the injection head 632 and withdrawing medicament from the vial 626 into the chamber 638 through the manifold. A gas manifold 650 is provided behind a proximal end 652 of the piston 646 for introducing gas from a line 654 behind the piston proximal end 652 in order to drive the piston 646 toward the casing distal end 618 thereby forcing medicament through the injection head 622.

In the embodiment 610, a cocking stud 656 may be provided in order to move the piston toward the casing proximal end 616 in order to withdraw medicament from the vial 626 and into the chamber 638 for subsequent injections.

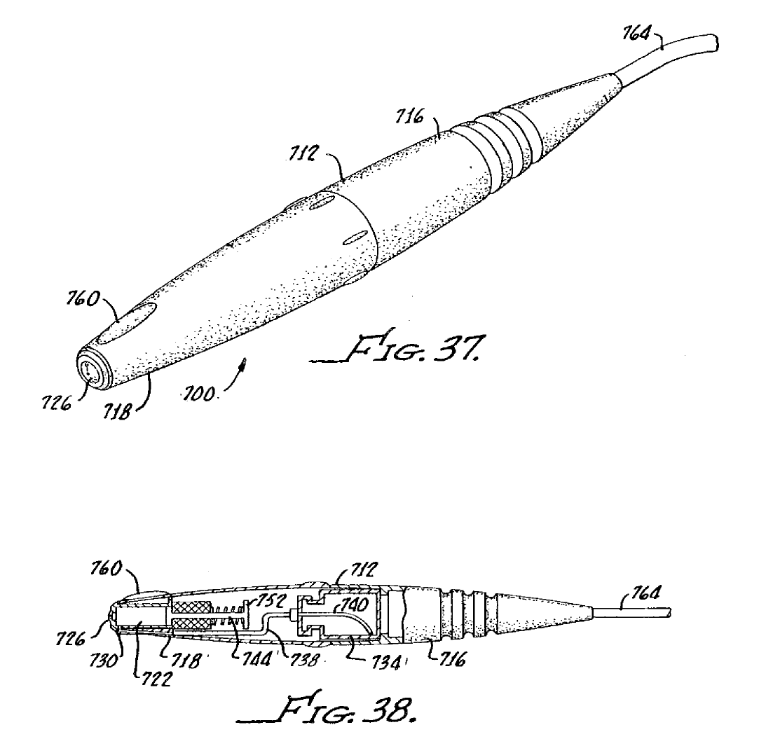

With reference to FIGS. 35 and 36, there is shown another embodiment 660 of an inline needleless injector in accordance with the present invention. Common reference characters represent identical or substantially similar elements, as hereinabove described in connection with the injector 610 as shown in FIGS. 33 and 34.

A manually operated gas valve 664, not shown, in FIGS. 33 and 34, is disposed proximate the casing 614 for enabling control of a gas through a gas line 668 into the manifold 650. In the embodiment 660, a spring 668 is provided as a means for moving the piston 646 toward the casing proximal end 616 after discharge of medicament through the injection head 622 in order to withdraw medicament from the vial 626 through the drug manifold 630.

Operation of the injector 660 is illustrated in FIGS. 35 and 36 respectively. In FIG. 35, the chamber which is filled with medicament, is ready for injection with the piston disposed with its proximal end 652 adjacent the gas manifold 650. Opening of the gas valve 664 causes gas to drive the piston toward the casing distal end forcing medicament through the injector 622, as shown in FIG. 36.

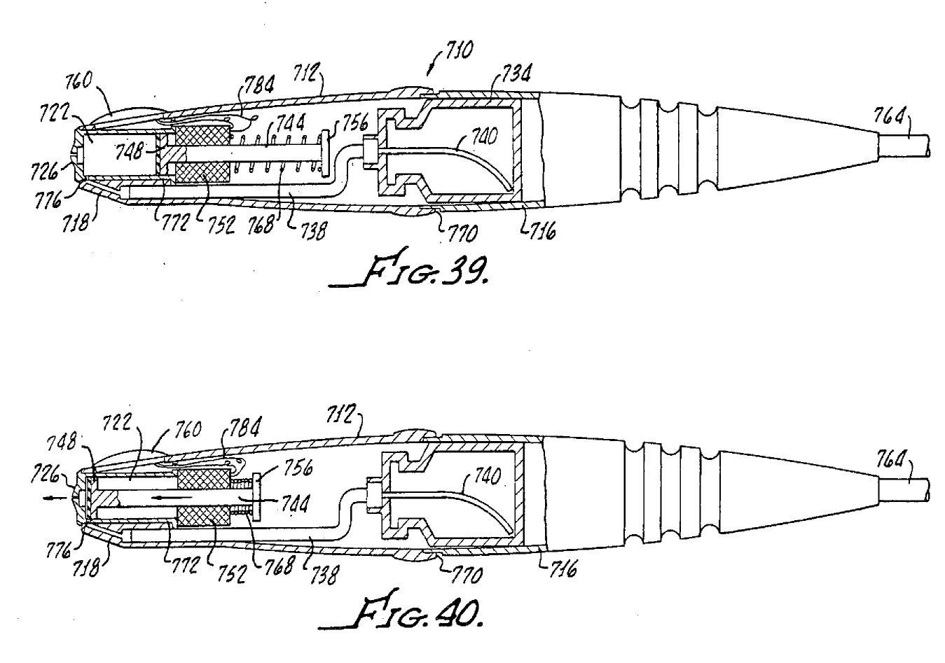

With reference to FIGS. 37 and 38, there is shown a eighth embodiment needleless injector 710 in accordance with the present invention including a housing 712 having a proximal end 716 and a distal end 718 with a cylinder 722 disposed in the housing 712 at the distal end 718 thereof along with an injection head 726 disposed at one end 730 of the cylinder 722. A vial 734 for containing a medicament, preferably BOTOX® (botulinum toxin), is disposed within the housing 712 at the proximal end 716 thereof. A manifold 738 interconnects the cylinder 722 with the vial 734 for transport of medicament from the vial 734 to the cylinder 722 utilizing a dip tube disposed within the vial 734.

A plunger 744 includes a first end, or piston, 748 slidably disposed within the cylinder 722 for forcing medicament in the cylinder 722 through the injection head 726 upon movement of the plunger 744 toward the injection head 726 and for withdrawing medicament from the vial 734 into the cylinder 722 upon movement of the plunger 744 away from the injection head 726. As hereinafter described, the plunger 744 is operable, or moveable, through the application of a magnetic flied provided by a solenoid stack 752. The solenoid 752 is disposed around the plunger 744 between the first end 748 and a second end 756 of the plunger 744. The solenoid 752 is disposed in an operational manner for forcing the plunger 744 toward the injection head 726 upon application of electrical current controlled by a trigger switch 760 interconnected with a power supply such as, for example, a 110 volt AC line 764.

A spring 768 is disposed around the plunger 744 between the solenoid 752 and the second end 756 for forcing a plunger 744 away from the injection head 726 after electrical current is not applied to the solenoid 752 in order to withdraw medicament from the vial 734 into the cylinder 722 as hereinabove noted.

As more clearly shown in FIGS. 39 and 40 for the vial 734 may be removable from the housing through a coupling 770 between the proximal end 716 and distal end 718 of the housing 712.

Operation of the device is illustrated in FIGS. 39 and 40, with FIG. 39 illustrating the injector 710 in a charged state with the plunger 744 disposed at a proximal end 772 of the cylinder 722.

Upon energizing the solenoid 752 through the application of electrical current controlled by the trigger switch 760, the plunger 744 is forced to a distal end 776 of the cylinder 722 as shown in FIG. 40 forcing medicament through the injector head 726 as indicated by the arrow 780. Electrical wiring 784, partially shown in FIG. 39, conventionally interconnects the power line 764, trigger 760 and solenoid 752.

After injection of medicament through the injector head 726, power is removed from the solenoid through the switch 760 enabling the spring 768 to force the plunger away from the injector head with the first end, or piston, 748 thereof withdrawing to the proximal end 772 of the cylinder 722.

Although there has been hereinabove described specific needleless injectors in accordance with the present invention for the purpose of illustrating the manner in which the invention may be used to advantage, it should be appreciated that the invention is not limited thereto. That is, the present invention may suitably comprise, consist of, or consist essentially of the recited elements. Further, the invention illustratively disclosed herein suitably may be practiced in the absence of any element which is not specifically disclosed herein. Accordingly, any and all modifications, variations or equivalent arrangements which may occur to those skilled in the art, should be considered to be within the scope of the present invention as defined in the appended claims.

What is claimed is:

1. A needleless injector comprising:
a barrel having a proximal and a distal end and a bore therethrough;
an injection head disposed at the barrel distal end in fluid communication with said bore;
a vial for containing a medicament, said vial being in fluid communication with said bore through a port near the barrel distal end;
a gas cartridge disposed at the barrel proximal end;
a bolt disposed in said bore for reciprocating movement therein, from a first position closing said port to a second position opening said port for release of medicament into said bore, said bolt being fitted to said bore in order to draw medicament from said vial into said bore upon movement of said bolt from the first position to the second position, and to force medicament through the injection head upon movement of the bolt from the second to the first position;
a cocking stud for enabling manual movement of said bolt to the second position;
a hammer disposed for reciprocal movement in said bore between said bolt and the barrel proximal end;
a mainspring disposed between said bolt and said hammer for forcing said hammer toward the barrel distal end;
a sear for releasably holding said bolt in the second position with mainspring compress between said bolt and said hammer;
a trigger disposed in an operational relationship with said rear for releasing the spring in order to drive the hammer toward the barrel distal end;
a valve tube disposed within said bore and having a distal end attached to said bolt and a proximal end, attached to the gas cartridge, said valve tube extending through said hammer;
a cup seal including a valve seat for controlling gas flow from the cartridge into said valve tube; and
a rear spring for releasably sealing the cup seal against said valve seat, said cup seal being opened momentarily by impact of said hammer to allow gas to flow through said valve tube to force said bolt to the first position in order to eject medicament through said injection head, said rear spring closing the cup seal agent the valve seal after said momentary opening.

2. A needleless injector comprising:
a barrel having a proximal and a distal end and a bore therethrough;
an injection head disposed at the barrel distal end in fluid communication with said bore;
a vial for containing a medicament, said vial being in fluid communication with said bore through a port near the barrel distal end;
a cylinder mounted for rotation at the barrel proximal end and having a plurality of chambers therein for receiving gas cartridges;
a frame for supporting said barrel and said cylinder; and
a bolt disposed in said bore for reciprocating movement from a first position closing said port to a second position opening said port for release of medicament into said bore, said bolt being fitted to said bore in order to draw medicament from said vial into said bore upon movement of said bolt from the medicament through the injection head upon movement of the bolt from the second to the first position.

3. The injector according to claim 2 first comprising a firing pin and a spring disposed adjacent said bolt for driving said bolt from the first to the second position.

4. The injector according to claim 3 further comprising a hammer disposed behind the bolt disposed for moving said gas cartridge, aligned with the hammer and bore, into the firing pin.

5. The injector according to claim 4 further comprising a trigger pivotably attached to said frame for moving the hammer in order to force the aligned cartridge into the firing pin for puncture of the aligned gas cartridge in order to drive the bolt from the second position to the first position and force medicament in said bore through said injection head, the spring upon release of pressure forcing the bolt from the first position to the second position and drawing medicament into the bore for repeated injection.

6. A needleless spring injector comprising:
a tubular casing having a bottom half shell extending from a rear end thereof;
a top half shell hingeably attached to the casing rear end for covering said bottom half shell;
a nose cap removably attached to a front end of said tubular casing;
an injection head disposed in an end of said nose cap;
a syringe replaceably disposed within said tubular casing and in fluid communication with said injection head;
a plunger slidably disposed within said tubular casing and engaging one end of said syringe for ejecting medicament therefrom through said ejection head;
a pushrod disposed between the bottom and top half shell for slidably moving said plunger;
a spring disposed about a rear portion of said pushrod for driving said pushrod;
a ratchet disposed on a forward portion of said pushrod;
a manually operated trigger, pivotably mounted within said tubular casing including a button extending outwardly from said tubular casing for manual depression thereof and a sear for releasably engaging said ratchet;
a cocking mechanism engaging said pushrod and said top half shell for compressing said spring upon hinged opening of said top half shell from said bottom half shell, said spring is retained in a compressed state by engagement of a said sear with said ratchet, whereby depression of said button releases said sear from said ratchet enabling expansion of said spring to drive said pushrod and said plunger in a forward direction in order to effect the ejection of said medicament from said syringe through said injection head.

7. A push-pull needleless injector comprising:
a housing having a distal and a proximal end;
a chamber disposed within said housing at the distal end thereof;
an injection head disposed at the housing distal end and in fluid communication with said chamber;
a vial for containing a medicament, said vial being in fluid communication with said chamber through a one-way valve;
a piston slidably disposed within said housing and having a piston head slidably disposed within said chamber and a stem disposed in the housing proximal end, said piston head being fitted to said chamber in order to draw medicament from said vial into said chamber through said one-way valve upon movement of said piston from a first position to a second position and to force medicament through the injection head upon movement of the piston from the second position to the first position;
a spring disposed around the piston stem for forcing said piston from the second position to the first position;
a cocking grip slidable disposed over the housing proximal end for compressing said spring;
a sear for releasably holding said piston in the second position with said spring compressed; and
a trigger disposed in an operational relationship with said sear for releasing said spring in order to drive said piston to said first position.

8. An inline needleless injector comprising:
a hollow casing having a proximal and a distal end;
an injection head disposed at the casing distal end;
a chamber disposed within the casing at the casing distal end;
a vial, for containing a medicament, disposed within the casing at the proximal end thereof;
a manifold for transport of the medicament from said vial to said chamber;
a one-way valve disposed in said manifold for preventing transport of medicament into said vial;
a piston slidably disposed within the chamber for forcing medicament through said injection head and withdrawing medicament from said vial into the chamber through said manifold;
a gas manifold disposed behind a proximal end of said piston for introducing gas behind the piston proximal end to drive said piston toward the casing distal end in order to force medicament through said injection head;
a valve in communication with said manifold for controlling gas introducing behind the piston proximal end; and
means for moving said piston toward the casing proximal end in order to withdraw medicament from said vial into the chamber through said manifold.

9. The injector according to claim 8 wherein said vial is removably disposed within the casing.

10. The injector according to claim 8 wherein the means for moving said piston toward the casing proximal end and comprises a cocking stud attached to said piston.

11. The injector according to claim 8 wherein the means for moving said piston toward the casing proximal end comprises a spring disposed around said piston.

12. A needleless solenoid driven injector comprising:
a housing having a proximal and a distal end;
a cylinder disposed in said housing at the distal end thereof;
an injection head disposed at one end of said cylinder;
a vial for containing a medicament disposed within said housing at the proximal end thereof;
a manifold interconnecting said cylinder and said vial for transport of medicament from said vial to said cylinder;
a one-way valve disposed in said manifold for preventing flow of medicament from said cylinder into said vial;
a plunger having a first end slidably disposed within said cylinder for forcing medicament in said cylinder through said injection head upon moving of said plunger toward said injection head and for withdrawing medicament from said vial and into said cylinder upon moving of said plunger away from said injection head, said plunger being moveable through the application of a magnetic field;
a solenoid disposed around the plunger between said first end and a second end of the plunger, said solenoid between disposed for forcing the plunger toward said injection head upon application of electrical current therethrough;
a trigger switch for controlling application of electrical current to said solenoid; and
a spring disposed around the plunger between said solenoid and said second end for forcing the plunger away from said injection head after electrical current is not applied to said solenoid in order to withdraw medicament from said vial into said cylinder through said manifold.

13. The injector according to claim 12 wherein said vial is removably disposed within said housing.
